(19) United States
(12) Patent Application Publication
(10) Pub. No.: US 2013/0116536 A1
(43) Pub. Date: May 9, 2013

(54) ACOUSTIC WAVE ACQUIRING APPARATUS AND ACOUSTIC WAVE ACQUIRING METHOD

(71) Applicant: CANON KABUSHIKI KAISHA, Tokyo (JP)

(72) Inventor: Akira Sato, Kawasaki-shi (JP)

(73) Assignee: CANON KABUSHIKI KAISHA, Tokyo (JP)

(21) Appl. No.: 13/644,383

(22) Filed: Oct. 4, 2012

(30) Foreign Application Priority Data
Nov. 4, 2011 (JP) 2011-242270

(51) Int. Cl. A61B 6/00

(52) U.S. Cl. 600/407

(57) ABSTRACT

Provided is an acoustic wave acquiring apparatus including: a probe configured to measuring acoustic waves that propagate from an object; scanning unit configured to moving the probe; region designating unit configured to receiving designation of a plurality of regions of interest in the object; and track determining unit configured to determining a plurality of designated measuring regions as regions for which the probe is to measure acoustic waves based on positions of the plurality of regions of interest, and determining a track upon moving the probe by the scanning unit based on positions of the plurality of designated measuring regions.

### BACKGROUND OF THE INVENTION

1. Field of the Invention

The present invention relates to an acoustic wave acquiring apparatus and an acoustic wave acquiring method.

2. Description of the Related Art

Conventionally, an ultrasound measuring apparatus of imaging the structure inside a biological object by transmitting ultrasound waves to the biological object and analyzing the reflected ultrasound waves has been put into practical application in the medical front. With an ultrasound measuring apparatus, when ultrasound waves are transmitted to the biological object, the reflection of ultrasound waves occurs at the interfaces in the biological object having different acoustic impedances. As a result of analyzing such reflected waves, the interfaces having difference acoustic impedances are imaged as configuration information in the biological object.

Moreover, in recent years, technology has been devised for analyzing the structure and condition of the surface and inside of a biological object by irradiating a laser beam onto the biological object and generating acoustic waves (photoacoustic waves) from the inside of the biological object based on such laser irradiation, and analyzing such photoacoustic waves (refer to Patent Literature 1 (PTL 1): U.S. Pat. No. 5,840,023). This technology is also referred to as photoacoustic wave measurement, and there is consideration for diverting this technology to medical use, such as for the examination of the inside of the human body, since examination can be performed non-invasively.

Both of the apparatuses described above are equipped with an acoustic probe for receiving the ultrasound waves (photoacoustic waves). As a configuration of an apparatus of using the acoustic probe as a detector, there are types which are handheld and used by being pressed against the skin near the region of interest which the user wishes to acquire information, and types which mechanically scan the surface of the skin of the biological object by introducing a mechanical scanning mechanism. Accordingly, measurement of the region of interest is being realized by providing the apparatus with manual or automatic scanning means.

With existing acoustic probes, it is difficult to produce a sensor with a large opening as with an X-ray imaging apparatus from the perspective of production yield and cost. Thus, the generally adopted method is to use an acoustic probe of a size that is smaller than the region that needs to be examined and covering such region to be examined via automatic or manual scanning.

A measuring apparatus which performs automatic scanning using an acoustic probe includes an input setting unit to be used by the user for setting the region of interest. The input setting unit is configured, for example, as a button input device such as a keyboard or a pointing device such as a mouse or a touch pen, and is used for inputting the detailed measurement setting using a keyboard or designating the measurement position using a pointing device such as a mouse. Among the foregoing apparatuses, there are types which enable the user to designate, in detail, the scanning track of the probe by using a touch pen or the like (refer to Patent Literature 2 (PTL 2): Japanese Patent Application Laid-Open No. 2006-000185).

PTL 1: U.S. Pat. No. 5,840,023

PTL 2: Japanese Patent Application Laid-Open No. 2006-000185

### SUMMARY OF THE INVENTION

As an example of designating the scanning track in a conventional apparatus, there is a method where a user operates a pointing device such as a touch pen and designates the scanning track while referring to the image of the object displayed on the display device. There are various methods of setting the scanning track and, for example, it is possible to draw a line as the track for moving the acoustic probe, or set the scanning track by designating a plurality of coordinates of the destination. The apparatus performs measurement while moving the acoustic probe so as to trace the designated scanning track.

Particularly when there area plurality of regions of interest, the user needs to be conscious of moving the acoustic probe upon making sure all regions of interest are included in the region to be measured when designating the scanning track, and the setting operation was complicated.

The present invention was devised in view of the foregoing problems, and an object of this invention is to provide technology capable of ameliorating the complication of the user's setting operation and improving the operation efficiency when designating a plurality of regions of interest and performing measurement using an acoustic probe.

The present invention provides an acoustic wave acquiring apparatus, comprising:

a probe configured to measure acoustic waves that propagate from an object;

scanning unit configured to move the probe;

region designating unit configured to receive designation of a plurality of regions of interest in the object; and

track determining unit configured to determine a plurality of designated measuring regions as regions for which the probe is to measure acoustic waves based on positions of the plurality of regions of interest, and determine a track upon moving the probe by the scanning unit based on positions of the plurality of designated measuring regions.

The present invention also provides an An acoustic wave acquiring method, comprising:

a step of measuring by a probe acoustic waves that propagate from an object;

a step of moving the probe by scanning unit;

a step of receiving by region designating unit designation of a plurality of regions of interest in the object; and

a step of determining by track determining unit a plurality of designated measuring regions as regions for which the probe is to measure acoustic waves based on positions of the plurality of regions of interest, and determining a track upon moving the probe by the scanning unit based on positions of the plurality of designated measuring regions.

According to the present invention, when designating a plurality of regions of interest and performing measurement using an acoustic probe, it is possible to provide technology capable of ameliorating the complication of the user's setting operation and improving the operation efficiency.

Further features of the present invention will become apparent from the following description of exemplary embodiments with reference to the attached drawings.

### BRIEF DESCRIPTION OF THE DRAWINGS

FIG. 1 is a configuration diagram of the photoacoustic measuring apparatus and the photoacoustic system operating apparatus according to the present invention;

FIG. 2 is a diagram showing an example of the region of interest setting screen;

FIG. 3 is a flowchart according to an embodiment of the present invention;

FIG. 4 is a schematic diagram of the setting of a plurality of regions of interest;

FIG. 5 is a schematic diagram of the inclusion region according to an embodiment of the present invention;

FIG. 6 is a diagram showing an example of the stripe according to an embodiment of the present invention; and

FIG. 7 is a schematic diagram of the determination of the scanning region according to an embodiment of the present invention.

### DESCRIPTION OF THE EMBODIMENTS

The present invention is now explained in further detail with reference to the drawings. As a general rule, the same constituent element is given the same reference numeral, and the explanation thereof is omitted. In the ensuing explanation, a photoacoustic apparatus is taken as an example of the acoustic wave acquiring apparatus of the present invention, but the application of the present invention is not limited thereto. The present invention can be applied to any apparatus which performs scanning using an acoustic probe according to the region of interest designated by the user and, for example, can also be applied to an ultrasound measuring apparatus which transmits ultrasound waves to an object and receives the reflected ultrasound echo for use in measurement.

### Embodiments

### (Configuration and Function of Photoacoustic Apparatus)

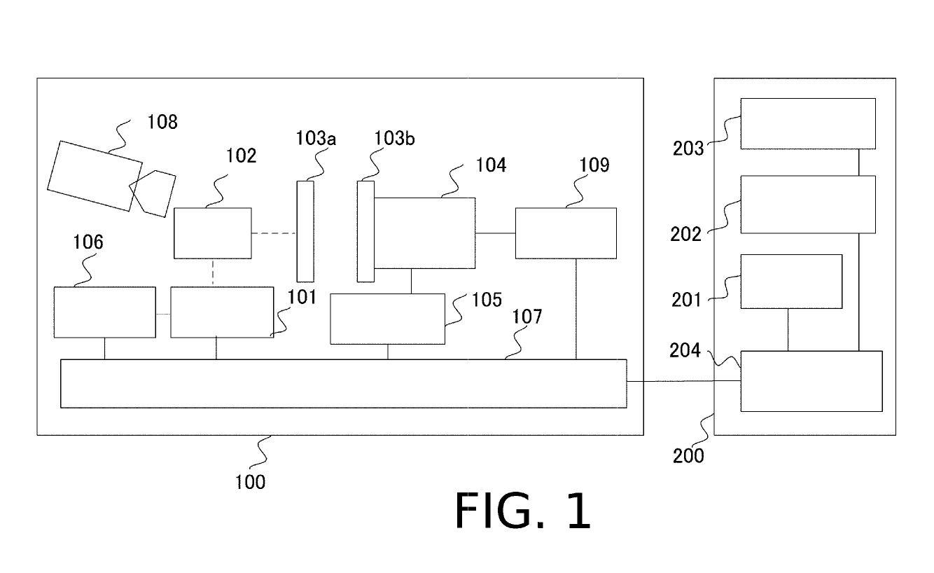

Foremost, the configuration of the photoacoustic apparatus according to this embodiment is now explained with reference to FIG. 1. The photoacoustic apparatus is an apparatus for acquiring internal information of an object. The photoacoustic apparatus is also sometimes configured as a photoacoustic imaging apparatus for imaging the acquired internal information of the object. When the object is a biological object, the photoacoustic apparatus enables the imaging of object information for the diagnosis of malignant tumors and vascular diseases or the follow-up of chemical treatment. The term “object information” in an apparatus that uses the photoacoustic effect as in this embodiment refers to the generation source distribution of the acoustic waves that were generated based on the irradiation of light, and shows the initial sound pressure distribution in the biological object or the light energy absorption density distribution derived therefrom. Moreover, substance concentration distributions such as oxygen saturation distribution and oxygenated and deoxygenated hemoglobin concentration distribution are also included therein. Meanwhile, object information in an ultrasound measuring apparatus using ultrasound echoes refers to the information which reflects the difference in the acoustic impedance of tissues in the object.

The photoacoustic apparatus comprises, as its main hardware configuration, a photoacoustic measuring apparatus 100 and a photoacoustic system operating apparatus 200. The photoacoustic measuring apparatus 100 comprises, as its hardware configuration, a laser light source 101, an optical system 102, an acoustic probe 104, an apparatus control unit 107, and a camera 108. The photoacoustic measuring apparatus 100 additionally includes a probe drive unit 105, a light source drive unit 106, and a signal processing unit 109. Moreover, the photoacoustic system operating apparatus 200 includes a region designating unit 201, an image generating unit 202, an image display unit 203, and a system control unit 204. Measurement of an object is now explained. An information processing apparatus such as a PC may be used as the photoacoustic system operating apparatus. However, the arrangement of the respective blocks is not limited to the illustrated example, and, for instance, all constituent elements may be formed integrally.

An object (not shown) such as a biological object is fixed by plates 103a, 103b for compressing and fixing the object from either side thereof. These plates are also sometimes referred to as compression plates. Pulsed light from a laser light source is guided to the surface of the plate 103a by the optical system 102 such as a lens, a mirror, or an optical fiber, becomes dispersed pulsed light, and irradiated on the object. When a part of the energy of light that propagated inside the object is absorbed by a light absorber such as blood vessels, acoustic waves are generated based on thermal expansion from that light absorber. In other words, the temperature of the light absorber increases pursuant to the absorption of the pulsed light, volume expansion occurs due to such temperature rise, whereby acoustic waves are generated. This phenomenon is referred to as a photoacoustic effect. Acoustic waves are a type of elastic waves, and include those which are referred to as sound waves, ultrasound waves, acoustic waves, photoacoustic waves, and optical ultrasound waves.

The acoustic probe 104 used for detecting acoustic waves is a detector configured from a plurality of receiving elements which detect acoustic waves. A detector detects the acoustic waves that were generated and propagated in the object, and converts such acoustic waves into an electric signal, which is an analog signal. This detection signal acquired from the detector is referred to as a “photoacoustic signal”.

The signal processing unit 109 acquires internal information of the object from the foregoing photoacoustic signal. The signal processing unit 109 amplifies the photoacoustic signal acquired from the acoustic probe 104 by using a reception amplifier, and converts this into a photoacoustic signal as a digital signal by using an A/D converter. In other words, a photoacoustic signal is a concept which includes both the analog signal detected by the acoustic probe and the digital signal that was processed by the signal processing unit. The photoacoustic signal as the digital signal is communicated to the photoacoustic system operating apparatus 200 based on the communication of the apparatus control unit 107 and the system control unit 204 via a communication line.

The image generating unit 202 generates an internal image of the object by performing arithmetic processing to the three-dimensional information based on image reconfiguration processing to the photoacoustic signal. The image display unit 203 displays the generated image (photoacoustic image) of the object. The system control unit 204 and the image generating unit 202 of the photoacoustic system operating apparatus 200 can be configured, for example, as a program or dedicated circuit that is operated by using the CPU resource. The same applies to the apparatus control unit 107, the signal processing unit 109 and the like of the photoacoustic measuring apparatus.

The camera 108 of the photoacoustic measuring apparatus 100 captures images of the object, and provides such images upon the user designating the region of interest of the object. Provision of images to the user can be performed, for example, by displaying such images on the image display unit 203.

The region designating unit 201 of the photoacoustic system operating apparatus 200 is means for receiving the designation of the region of interest by the user who viewed the images captured by the camera 108.

The main constituent elements and the respective components requiring a detailed explanation are now explained in order.

### (Laser Light Source 101)

When the object is a biological object, irradiated from the light source is light of a specific wavelength that is absorbed by a specific component among the components configuring the biological object. As the light source, preferably used is a pulsed light source capable of generating pulsed light in the order of several nanoseconds to several hundred nanoseconds. While laser is preferable as the light source, a light-emitting diode or the like may also be used in substitute for a laser. As the laser, solid-state laser, gas laser, dye laser, semiconductor laser and other lasers may be used.

Note that, in this embodiment, while a single light source is shown as an example, a plurality of light sources may also be used. In the case of using a plurality of light sources, a plurality of light sources that oscillate the same wavelength may be used in order to increase the irradiation intensity of the light to be irradiated on the biological object, or a plurality of light sources having a different oscillation wavelength may be used in order to measure the difference in the wavelength of optical characteristic value distribution. Note that, as the light source, if it is possible to use dyes or optical parametric oscillators (OPO) capable of converting the oscillation wavelength, it is also possible to measure the difference in the wavelength of optical characteristic value distribution.

With respect to the wavelength to be used by the laser light source, a wavelength region of 700 nm to 1100 nm, with low absorption in the biological object, is preferably used. However, when obtaining the optical characteristic value distribution of the biological object tissue which is relatively near the biological object surface, a wavelength region that is broader than the foregoing wavelength region; for instance, a wavelength region of 400 nm to 1600 nm may also be used.

Moreover, the irradiation frequency is usually predetermined for a laser light source. This is set forth as a design value for the sake of continuous irradiation of pulsed light of the intended intensity. However, the higher the irradiation frequency is the better since the irradiation frequency affects the number of photoacoustic measurements that can be performed per unit time. In this embodiment, the irradiation frequency of the laser light source is 10 Hz.

### (Acoustic Probe 104)

An acoustic probe is a detector which detects acoustic waves and converts the detected acoustic waves into an electric signal. The photoacoustic waves generated from an object are ultrasound waves of 100 KHz to 100 MHz. Thus, as the acoustic probe 104, a detector capable of receiving the foregoing frequency band is used. Any detector may be used so as long as it is able to detect the acoustic wave signal; for instance, a transducer that uses the piezoelectric phenomenon, a transducer that uses the oscillation of light, or a transducer that uses the change in capacity. The acoustic probe 104 of this embodiment is preferably configured from a detector in which a plurality of receiving elements are arrayed two-dimensionally. As a result of using such two-dimensional arrayed elements, acoustic waves can be simultaneously detected at a plurality of locations and, therefore, it is possible to shorten the detection time as well as reduce the influence of vibration of the object and so on. In this embodiment, let it be assumed that the receiving element pitch is a 2 mm interval, the receiving element array is 5 elements in the main scanning direction, and 5 elements in the sub scanning direction. Note that the main scanning direction is a direction that the acoustic probe moves while receiving acoustic waves from the object, and the sub scanning direction is a direction that is orthogonal to the main scanning direction for moving the acoustic probe. However, there are also regions where acoustic waves do not need to be received as with the moving region described later.

### (Camera 108)

With the photoacoustic apparatus of this embodiment, a camera is installed for providing images to be referred to upon designing the regions of interest to be subject to photoacoustic measurement. The camera is installed in a visual line direction that is substantially orthogonal to the holding plates that compress and hold the object, and the captured image is transmitted to the photoacoustic system operating apparatus. The visual field of the camera is preferably installed at a view angle in which the photoacoustic measurable range can be viewed. The camera is installed so that the compressed and held object can be observed, and the user can designate the region of interest while observing the compressed and held object. However, even in cases where the imaging direction of the camera is not substantially orthogonal to the hold plates due to restrictions of the apparatus, it is possible to display the overall image to be viewable by the user based on image correction or the like.

### (Region Designating Unit 201)

The photoacoustic apparatus of this embodiment includes means for the user to designate the region of interest. The user designates the region of interest by using input unit such as a mouse while referring to the observed image of the compressed and held object that is displayed on the display device. The input unit is not limited to a mouse or a keyboard, and may also be a pen tablet type or a touch pad mounted on the display device surface. In this embodiment, a plurality of regions of interest can be designated.

With the photoacoustic measurement, since the photoacoustic waves that propagated from the inside of the object are also acquired as a signal, in addition to a two-dimensional sliced image, it is also possible to perform imaging of a three-dimensional object. Nevertheless, in this embodiment, the user will designate a two-dimensional rectangle. Moreover, while a rectangular range is designated in this embodiment for ease of understanding, the shape is not limited thereto. The user sets the location corresponding to the position to be subject to photoacoustic measurement with a two-dimensional rectangle on the image plane while referring to the observed image from a specific direction of the object. The two-dimensional rectangular range that was designated by the user for measurement based on the region of interest is referred to as a measurement designated region. In other words, the user will set the measurement designated region while referring to the observed image from a specific direction of the object and envisaging that the depth portion as the inside thereof being measured and imaged.

As the method of designating the measurement region, coordinates may also be designated based on input using a keyboard. The coordinate designating method in the foregoing case may be the designation of central coordinates of the measurement region of a predetermined size in order to specify the measurement region, or a plurality of vertex coordinates may be designated on the reference image plane so as to set the measurement designated region. The designation of the vertex coordinates may be a designation of two points on a diagonal line in the case of a rectangular range. In all of the foregoing cases, it is possible to set a measurement designated region as the two-dimensional rectangular region on the reference image plane.

The photoacoustic apparatus that received the designation from the user converts the image coordinate system of the image captured by the camera into the apparatus coordinate system based on the measurement designated region. In addition, by controlling the probe drive unit 105 and the light source drive unit 106, the probe and the laser light source (measurement system) on the actual object are moved. The probe drive unit corresponds to the scanning unit of the present invention.

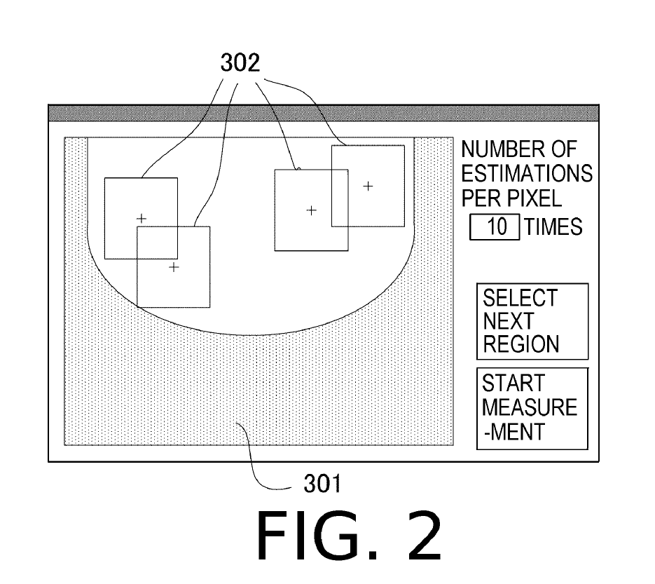

The screen image for designating the region in this embodiment is shown in FIG. 2. In the diagram, 301 represents an observed image from a specific direction relative to the object, and 302 represents a measurement designated region that was designated by the user while referring to the observed image. The measurement designated region 302 may be designated in a pre-set size. Moreover, by inputting a rectangle with a pointing device, it is also possible to designate a measurement region of an arbitrary size. Moreover, a function for designating a plurality of measurement designated regions is also provided. For example, this is a method where a multiple selection button is provided and, when the measurement designated region is designated while pressing the multiple selection button, a plurality of measurement designated regions which were selected while the multiple selection button is being pressed are stored. As another method, by providing a “Select next region” menu on the menu screen and designating this menu each time a measurement designated region is designated, the region of interest can be designated successively. In all of the foregoing methods, it is preferable to prepare means for cancelling a part or all of the designations of the measurement designated region.

### (Measurement Flow)

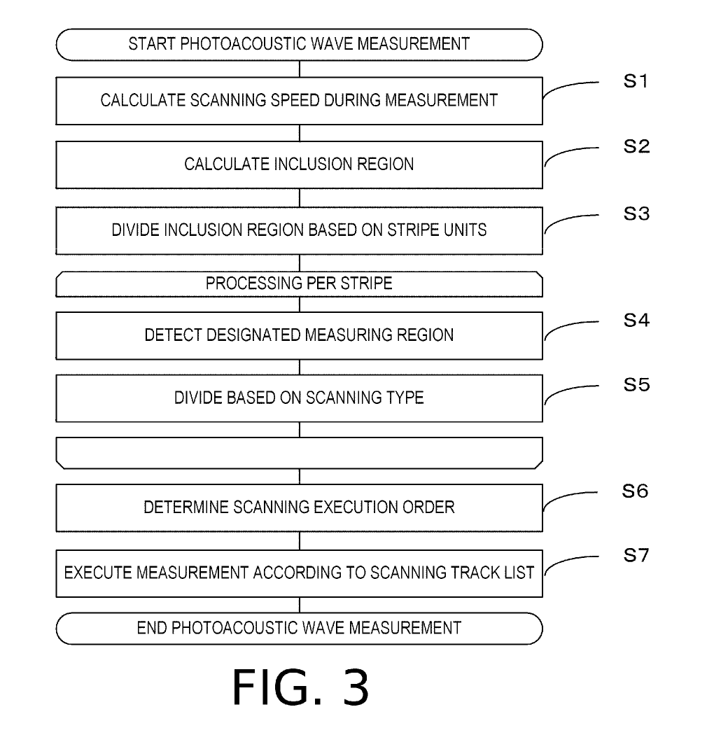

The actual measurement is now explained with reference to the flowchart of FIG. 3. This flow is based on the premise that the measurement designated region has been designated by the user, and is started with the instruction for starting photoacoustic measurement as the trigger.

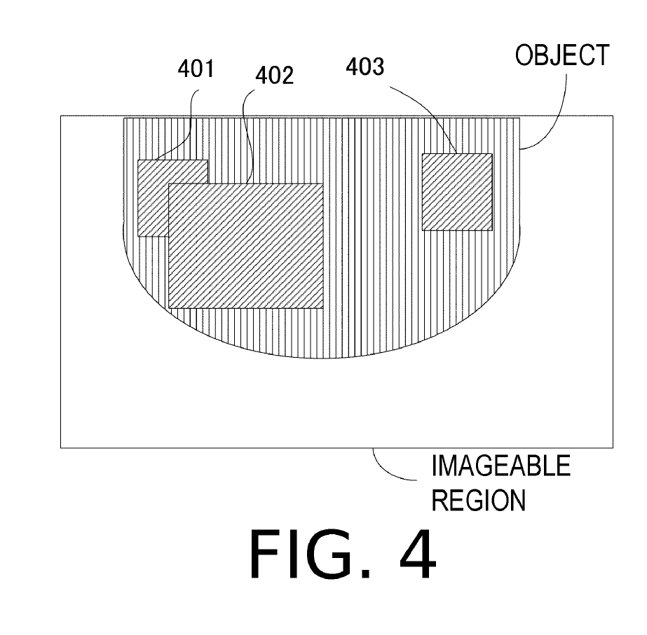

The measurement designated region that was designated is foremost explained with reference to FIG. 4. In FIGS. 4, 401, 402, and 403 represent the measurement designated region that was designated by the user via the region designating unit 201. Here, it is also possible to set the measurement conditions of the photoacoustic measurement. In this embodiment, let it be assumed that the number of acquisitions (cumulative number) of the photoacoustic data per pixel is set to 10 times. In accordance with the cumulative number, effects of being able to improve the SN ratio of the acquired object information can be obtained.

After setting the measurement designated region, triggered by the instruction from the user for starting measurement, communication is made from the system control unit 204 of the photoacoustic measurement system controller 200 to the apparatus control unit 107 of the photoacoustic measuring apparatus 100, and the start of photoacoustic measurement is requested.

### (Calculation Process of Scanning Speed for Measurement)

When the apparatus control unit 107 receives a measurement start request message, the apparatus control unit 107 foremost calculates the scanning speed (step S1 of FIG. 3). Here, let it be assumed that the number of elements of the acoustic probe in the main scanning direction is Enx elements, the element pitch is Ep (mm), the cumulative number of photoacoustic measurement is Mn times, the light-emitting frequency of the laser light source is LHz (Hz). In order to simplify the explanation, let it be assumed that the cumulative number Mn is a multiple of the number of elements Enx. Here, the scanning speed Vx (mm/sec) of the measurement system (acoustic probe and laser light source) in the main scanning direction is calculated based on Formula (1), and the number of scans Sn is calculated based on Formula (2), respectively.

Vx=Ep×LHz (1)

Sn=Mn/Enx (2)

In the case of this embodiment, since the number of elements Enx of the acoustic probe 104 in the main scanning direction is 5 elements, and the cumulative number Mn is set to 10 times, when the acoustic probe 104 is to be moved for each receiving element at a time, the number of scans Sn is calculated as 2 based on Formula (2). In other words, if the acoustic probe makes one full round, 10 estimations can be performed. Moreover, since the element pitch Ep is 2 mm and the light-emitting frequency LHz of the laser light source is 10 Hz, based on Formula (1), the scanning speed Vx of the measurement system upon measurement will be 20 (mm/sec).

The scanning speed Vx and the number of scans Sn obtained as described above are used for calculating the scanning region explained later and determining the measurement order. The apparatus control unit in the foregoing case corresponds to the speed calculation unit of the present invention.

Note that, when an ultrasound measuring apparatus is to be used rather than a photoacoustic apparatus, the scanning speed and number of scans of the main scanning direction for realizing the cumulative number can be determined according to the capability of the beam formation, rather than the light-emitting frequency. In other words, the scanning speed of the probe can be calculated based on the drive frequency and element pitch of the acoustic probe.

For more complex conditions; for example, when the cumulative number Mn is smaller than the number of elements Enx in the main scanning direction, or a multiple of a value that is smaller than Enx, the cumulative number per a full round of probe movement will decrease. In the foregoing case, the scanning speed can be set high if scanning is performed while shifting two or more pixels per unit time upon determining the displacement of the acoustic probe. The moving speed of the acoustic probe is not limited to the method illustrated in this embodiment, and various algorithms for adjusting the scanning speed may be applied in dependence of the measurement conditions or apparatus configuration.

The scanning speed calculation function in this embodiment aims to obtain the probe moving speed for measurement and, therefore, the reference parameters and algorithms are not limited to those described in this embodiment.

### (Calculation of Inclusion Region)

Subsequently, the apparatus control unit 107 calculates the inclusion region which includes all of the plurality of measurement designated regions that are designated (step S2). Among the plurality of measurement designated regions, the measurement designated region positioned at the outermost shell is specified, and the rectangle that circumscribes the measurement designated region of the outermost shell is obtained as the inclusion region.

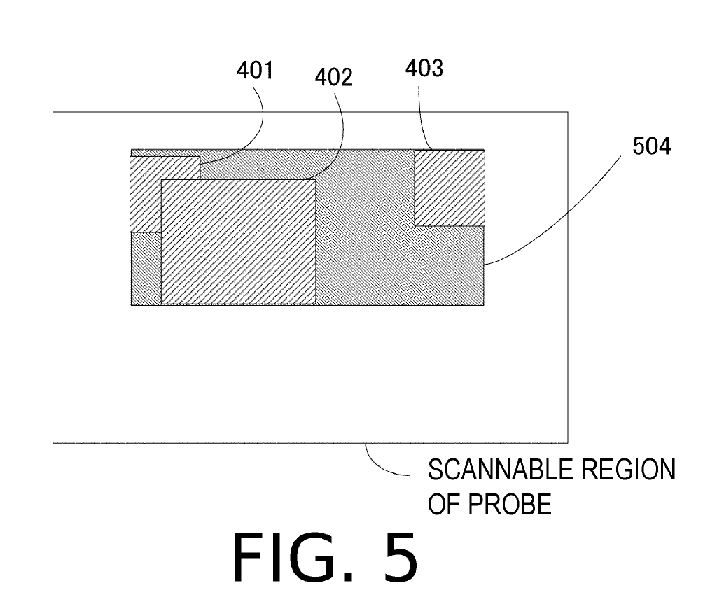

FIG. 5 shows an image of the inclusion region. In FIG. 5, the rectangle 504 is the inclusion region calculated from the measurement designated regions 401, 402, and 403. When the measurement designated regions are rectangular as described above, the inclusion region is the region obtained by extending the respective sides of all measurement designated regions and selecting, as a rectangle, the outermost sides in the vertical and horizontal directions.

### (Processing per Stripe)

Subsequently, the apparatus control unit 107 divides the inclusion region into regions based on stripe units (step S3). Here, a stripe refers to the region to be subject to photoacoustic measurement by moving the acoustic probe and the laser light source (together, also referred to as the “measurement system”) in the main scanning direction. In this embodiment, the size that a photoacoustic signal can be obtained with a single laser emission is the size of all element regions of the acoustic probe. In reality, while the region subject to photoacoustic measurement is a three-dimensional region including the depth direction, unless separately provided for herein, the plane in which the region subject to photoacoustic measurement is cut out from a plane that is parallel to the scanning of the measurement system is indicated as a “stripe”.

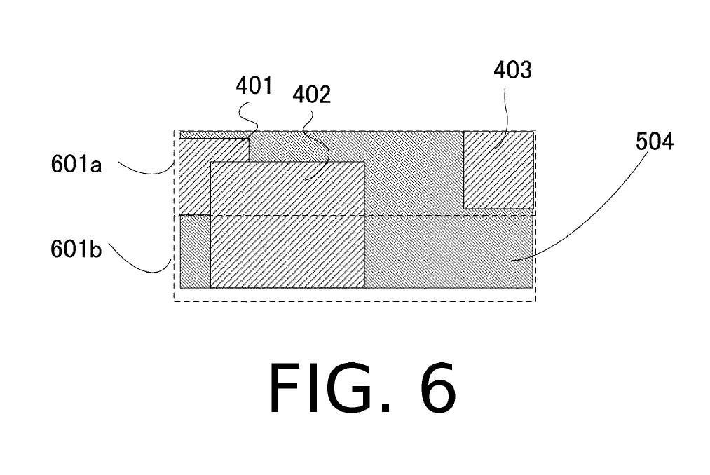

FIG. 6 shows an example of the division into regions based on a stripe size. 601a, 601b are respectively divided regions of the inclusion region 504 in stripe units.

Next, for each region that was cut out in stripe units, the region (designated measuring region) that needs to be measured without fail since it contains the measurement designated region designated by the user is detected (step S4). Here, contact of a notable stripe and the measurement designated region is determined, and the overlapping portion of the two is determined to be the designated measuring region.

In addition, here, even if the portion where the notable stripe and the measurement designated region are in contact is a part of the region (stripe height) of the entire width of the stripe in the sub scanning direction, the region of the stripe height portion is determined as the designated measuring region. For example, in FIG. 6, the portion where the stripe 601a and the measurement designated region 401 is overlapping is only for the height of the measurement designated region 401, and is lower than the height of the stripe 601a. Nevertheless, in this case also, the height of the designated measuring region is the height of the stripe 601a. Moreover, there are also cases where a range such as the lower part of the stripe 601b, which is not included in the inclusion region, falling within the designated measuring region.

Subsequently, the apparatus control unit 107 divides the notable stripe into three types of scanning regions according to the type of scanning (step S5). Here, determination processing of determining whether the regions in the stripe are any one of the scanning regions of the following three types. Each scanning region uniformly becomes a unit for performing the processing of photoacoustic measurement or movement.

(1) First is the continuous scanning region of performing photoacoustic measurement while causing the measurement system to perform continuous scanning.

(2) Second is the moving region which only moves the measurement system.

(3) Third is the fixed measuring region to be measured upon stopping the measurement system.

Upon making the foregoing determination, whether the designated measuring region is to be measured via continuous scanning or measured via repeated fixed measuring is determined. Moreover, when there are a plurality of designated measuring regions, whether the region between the designated measuring regions is to be a moving region or a continuous scanning region is determined. The criteria for determination is to determine the region so that the time required for the respective methods and the time required for the measurement of the notable stripe and movement processing will be shortened. The continuous scanning time is calculated from the foregoing continuous scanning speed Vx and scanning distance, the fixed scanning time is calculated from the cumulative number and laser irradiation frequency, and the simple moving time is calculated from a predetermined design value. The apparatus control unit in the foregoing case corresponds to the track determining unit of the present invention.

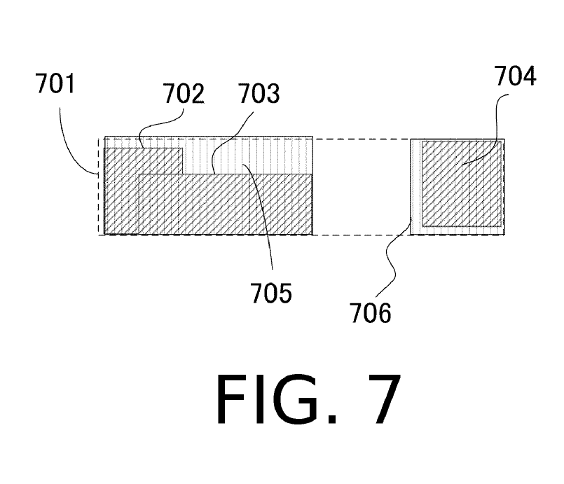

An example of the determination result of the region division processing in the notable stripe is shown in FIG. 7. In the diagram, 701 represents the notable stripe. 702, 703, and 704 are regions that are in contact with the notable stripe 701 among the measurement designated regions 401, 402, and 403 that were designated by the user. Here, as an example of dividing the stripe, it is possible to divide the stripe, in the main scanning direction, to be a portion in which only 702 overlaps with the stripe, a portion in which 702 and 703 overlap, a portion in which only 703 overlaps, a portion in which none of them overlaps, and a portion in which only 704 overlaps. Otherwise, more simply, it is also possible to divide the stripe, in the main scanning direction, a portion which overlaps with any one of the measurement designated regions, and a portion which does not overlap with any one of the measurement designated regions. In all of the foregoing dividing methods, the height of the designated measuring region is the height of the stripe.

In the example of this diagram, with respect to the designated measuring region including the measurement designated regions 702, 703, the time required for performing continuous scanning and the time required for repeatedly performing fixed measuring are compared, and these are set as a continuous scanning region 704 to be subject to continuous scanning at once. Here, when repeating the fixed measuring, the moving time between the fixed measuring regions will be required in addition to the time of photoacoustic measurement corresponding to the set cumulative number. Moreover, since the width of the fixed measuring region is fixed, caution must be given to the possibility that a portion (for instance, the moving region) that is not included in the designated measuring region could be measured.

Meanwhile, the designated measuring region including the right-side measurement designated region 704 is set as a fixed measuring region 706. As illustrated in the diagram, based on the width of the acoustic probe, since the width of the region 704 in the main scanning direction is included in the width of the fixed measuring region 706 (that is, the width of the element surface of the acoustic probe), continuous scanning is not required.

In addition, the remaining portion that was not included in either the continuous scanning region 705 or the fixed measuring region 706 is set as a moving region.

The foregoing processing of steps S4 and S5 is repeated for all stripes, and the region division of the entire inclusion region is performed.

### (Determination of Scanning Track)

When the region division processing of all stripes is complete, the apparatus control unit 107 determines the execution order of measurement regarding the respectively divided scanning regions (step S6). The criteria for determination is to shorten the total measurement time. Specifically, the execution order is determined so as to shorten the movement distance of the measurement system; particularly the movement distance outside measurement. For example, the mutual distance of the designated measuring regions detected in step S4 is obtained, and measurement may be performed in the order from the shortest movement distance. For instance, when starting the measurement from the upper left in the inclusion region of this embodiment, scanning is foremost started from the continuous scanning region 705, and the scanning region that is near the end point of 705 is subsequently measured.

Subsequently, a measuring list track is created according to the execution order. The measuring track list stores information listed in the measurement execution order with, at least, the measurement start coordinates, measurement information (continuous scanning measuring or fixed measuring), information regarding the scanning distance in the case of continuous scanning as one set.

The photoacoustic apparatus refers to the measuring track list that was finalized based on the foregoing procedure, moves the measurement system and acquires acoustic waves by controlling the light source drive unit 106 and the probe drive unit 105, and thereby performs measurement (step S7).

As explained above, the photoacoustic apparatus of this embodiment divides the measurement target into regions so as to include all of the plurality of measurement designated regions that were designated on the reference image, automatically determines the track (measurement execution order) on which the measurement system is to be moved, and thereby performs photoacoustic measurement. Consequently, the user is no longer required to be conscious of the scanning track, and an improved apparatus which allows the user to concentrate on designating the region of interest is thereby provided.

Moreover, the photoacoustic apparatus of this embodiment calculates the continuous scanning speed based on the measurement condition setting, divides the scanning region with the time required for the measurement as the measurement condition, and calculates the scanning track with the movement distance other than measurement as the determination condition. Consequently, it is possible to calculate the track which enables efficient scanning based on the plurality of measurement designated regions that were designated, and thereby shorten the measurement time.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2011-242270, filed on Nov. 4, 2011, which is hereby incorporated by reference herein its entirety.

What is claimed is:

1. An acoustic wave acquiring apparatus, comprising:
a probe configured to measure acoustic waves that propagate from an object;
scanning unit configured to move the probe;
region designating unit configured to receive designation of a plurality of regions of interest in the object; and
track determining unit configured to determine a plurality of designated measuring regions as regions for which the probe is to measure acoustic waves based on positions of the plurality of regions of interest, and determine a track upon moving the probe by the scanning unit based on positions of the plurality of designated measuring regions.

2. The acoustic wave acquiring apparatus according to claim 1,
wherein regions included in the plurality of regions of interest are included in any of the plurality of designated measuring regions.

3. The acoustic wave acquiring apparatus according to claim 1, wherein
the scanning unit moves the probe in a main scanning direction and a sub scanning direction which is orthogonal to the main scanning direction in an inclusion region which is a region containing all of the plurality of regions of interest, and
the track determining unit divides a stripe, which is a rectangular region formed by the probe moving in the main scanning direction, into a region which includes the region of interest and a region which does not include the region of interest, and uses the region which includes the region of interest as the designated measuring region.

4. The acoustic wave acquiring apparatus according to claim 3,
wherein the designated measuring region includes a continuous scanning region for measuring acoustic waves while moving the probe by the scanning unit, and a fixed measuring region for measuring acoustic waves by stopping the probe.

5. The acoustic wave acquiring apparatus according to claim 4,
wherein the track determining unit determines the track so as to shorten a measurement time configured from a time required for movement of and measurement by the probe in the continuous scanning region, a time required for measurement by the probe in the fixed measuring region, and a time required for movement of the probe between the plurality of designated measuring regions.

6. The acoustic wave acquiring apparatus according to claim 5,
wherein the track determining unit determines an order of measuring the plurality of designated measuring regions so as to shorten a movement distance of the probe, and determines the track in use the order.

7. The acoustic wave acquiring apparatus according to claim 5, further comprising:
speed calculation unit configured to calculate a moving speed of the probe in the continuous scanning region based on information at least related to elements of the probe,
wherein the track determining unit obtains the time required for the movement of and measurement by the probe in the continuous scanning region in use of the calculated speed.

8. The acoustic wave acquiring apparatus according to claim 7, wherein
acoustic waves that propagate from the object are photoacoustic waves that are generated from the object irradiated with light, and
the speed calculation unit calculates the moving speed from a frequency of the light and an element pitch of the probe in the main scanning direction.

9. The acoustic wave acquiring apparatus according to claim 7, wherein
acoustic waves that propagate from the object are reflections of the acoustic waves transmitted by the probe to the object, and
the speed calculation unit calculates the moving speed from a drive frequency of the probe and an element pitch of the probe in the main scanning direction.

10. The acoustic wave acquiring apparatus according to claim 1, further comprising:
input unit configured to receive input from a user,
wherein the region designating unit displays an image of the object on a display device, and receives a designation of the region of interest by means of the input unit from the user referring to the image of the object.

11. The acoustic wave acquiring apparatus according to claim 10,
wherein the designation of the region of interest by the user using the input unit is performed by designating a two-dimensional rectangular region.

12. The acoustic wave acquiring apparatus according to claim 10, further comprising:
a camera configured to acquire an image of the object,
wherein the region designating unit displays the image acquired by the camera on the display device.

13. An acoustic wave acquiring method, comprising:
a step of measuring by a probe acoustic waves that propagate from an object;
a step of moving the probe by scanning unit;
a step of receiving by region designating unit designation of a plurality of regions of interest in the object; and
a step of determining by track determining unit a plurality of designated measuring regions as regions for which the probe is to measure acoustic waves based on positions of the plurality of regions of interest, and determining a track upon moving the probe by the scanning unit based on positions of the plurality of designated measuring regions.
